(19) United States
(12) Patent Application Publication
(10) Pub. No.: US 2023/0073767 A1
(43) Pub. Date: Mar. 9, 2023

(54) SYSTEM AND METHOD FOR COLLECTING AND SENSING VOLATILE COMPOUNDS

(71) Applicant: NANOSCENT LTD., Misghav (IL)

(72) Inventor: Yair PASKA, Kiryat Ata (IL)

(73) Assignee: NANOSCENT LTD., Misghav (IL)

(21) Appl. No.: 17/799,120

(22) PCT Filed: Feb. 11, 2021
(86) PCT No.: PCT/IL2021/050164

§ 371 Date: Aug. 11, 2022

Related U.S. Application Data
(60) Provisional application No. 62/975,920, filed on Feb. 13, 2020.

(51) Int. Cl. G01N 33/00

(52) U.S. Cl. CPC G01N 33/0047

(57) ABSTRACT

Systems and methods are provided for collecting and sensing volatile compounds (VCs). Systems may comprise a collecting unit for collecting a source of VCs; a container for containing the collected source of VCs; one or more scent recorders comprising at least one sensor for detecting VCs; and a delivery unit for delivering a gas comprising VCs collected from the source, via the container—towards the one or more scent recorders. Optionally, a first portion of the delivered gas comprising the VCs may be used for regenerating the one or more scent recorders, and a second portion of the delivered gas comprising the VCs may be used for sensing the VCs by the one or more scent recorders.

### BACKGROUND OF THE INVENTION

### 1. Technical Field

The present invention relates to the field of sensing volatile compounds, and more particularly, to collecting and sensing of volatile compounds.

### 2. Discussion of Related Art

Volatile compounds (VCs) can be sensed and used to diagnose health conditions in humans and animals as well as VCs in various industrial facilities. For example, VCs can be used for detecting the presence of bacteria and viruses in humans and animals using specific VCs they discharge during their lifecycles. Specific VCs can be collected and identified using scent recorders (also known in the art as electronic noses) and provide a relatively simple and quick measurement method.

Unlike known diagnosis methods that require expensive and/or complicated laboratory equipment and are time consuming, the use of VCs is simple and the diagnosis is received almost instantly (in matter of seconds).

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a system for collecting and sensing volatile compounds (VCs), the system comprising: a collecting unit for collecting a source of VCs; a container for containing the collected source of VCs; one or more scent recorders comprising at least one sensor for detecting VCs; and a delivery unit for delivering a gas comprising VCs collected from the source, via the container—towards the one or more scent recorders. Optionally, a first portion of the delivered gas comprising the VCs is used for regenerating at least part of the one or more scent recorders, and a second portion of the delivered gas comprising the VCs is used for sensing the VCs by the one or more scent recorders.

One aspect of the present invention provides a method of collecting and sensing volatile compounds (VCs) by a corresponding system, the method comprising: collecting a source of VCs and accumulating gas comprising the VCs; delivering a first portion of the gas comprising the VCs for regenerating at least part of the system; delivering a second portion of the gas comprising the VCs to one or more scent recorders that comprise at least one sensor for detecting VCs; and sensing the VCs in the second portion of the gas using the one or more scent recorders, wherein the one or more scent recorders is regenerated prior to or after the sensing.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1A is a high-level schematic block diagram of a system for collecting and sensing volatile compounds according to some embodiments of the invention.

FIG. 1B is a high-level schematic illustration of a delivery system, according to some embodiments of the invention.

FIG. 2A is a high-level schematic illustration of a scent recorder, according to some embodiments of the invention.

FIG. 2B is a high-level schematic illustration of a chemiresistor sensor, according to some embodiments of the invention.

FIG. 2C is a high-level schematic illustration of a particle in a chemiresistor sensor, according to some embodiments of the invention.

FIGS. 3A-3D are high-level schematic illustrations of inflatable containers and collecting units for collecting a gas source of VCs, according to some embodiments of the invention.

FIG. 4A is a high-level schematic illustration of a container and a collecting unit for collecting liquids or solids, according to some embodiments of the invention.

FIG. 4B is a high-level schematic illustration of a collecting unit for collecting liquids or solids, according to some embodiments of the invention.

FIG. 5 is a high-level schematic flowchart of a method of collecting and sensing volatile compounds, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, to where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Some aspects of the invention are directed to a system and method or collecting and sensing VCs, for example, from patients (either human or other animals). A system according to embodiments of the invention may include one or more scent recorders (also known as electronic noses) for sensing the VCs. A system according to embodiments of the invention may collected VCs from liquids, solids or gas originated form the patient. In some embodiments, if originated from liquids or solids, the system may flush a source (e.g., solid and/or liquid) of VCs with a carrier gas for collecting the VCs. The gas carrying the VCs (either as originated or as a result of flushing) may be used for dual purpose, regenerating the one or more scent recorders following by sensing the presence of the VCs, for example, from prior use. It is noted that the term “scent” used herein relates to volatile compounds (VCs) in general, not necessarily to VCs that are detectable by humans. Disclosed scent detectors may be applied to measure any type of VC, with corresponding configuration of disclosed sensor(s).

Advantageously, disclosed embodiments overcome challenges in using VCs as a diagnostic tool, such as the collection of the VCs from humans and/or animals and the delivery of the collected VCs to the scent recorder(s) for analysis. Furthermore, disclosed embodiments provide accurate readings, reducing noise coming from undesired VCs and humidity, and also provide solutions for cleaning the sensing system. Disclosed embodiments include a simple and reliable collecting and sensing system that may allow using VCs originated from humans and animals as a diagnostic tool.

Currently known methods for regenerating scent recorders usually include flushing the scent recorder by “clean” gas in order to evacuate any previously trapped VCs from the sensors of the scent recorders. In an unexpected way, it was found that flushing or cleaning the scent recorder with a “contaminated” gas containing VCs to be analyzed following the flushing, did not harm and even increase the accuracy of the analysis, in comparison to standard regeneration methods.

As used herein the term “volatile compound” (VC) may be related to any volatile material, either organic or inorganic compound. Some examples for VCs may include: Cr₂, O₂, H₂, CH₄, NH₃, CH₃OH, SOₓ, NOₓBTEX, Benzene, Toluene, Ortho-Xylene, Para-Xylene, Meta-Xylene and Ethylbenzene and the like.

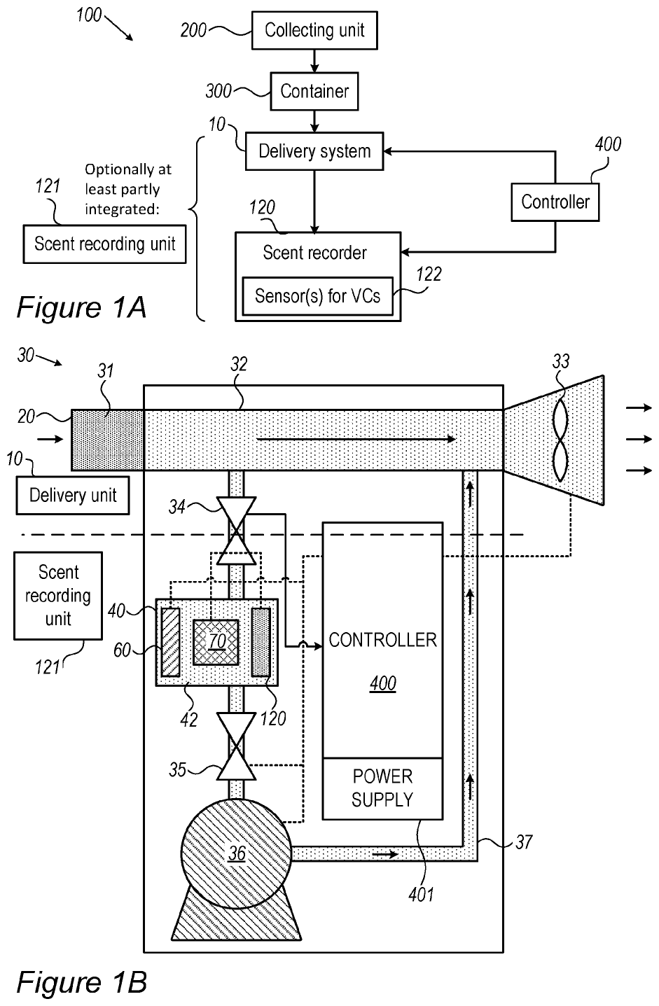

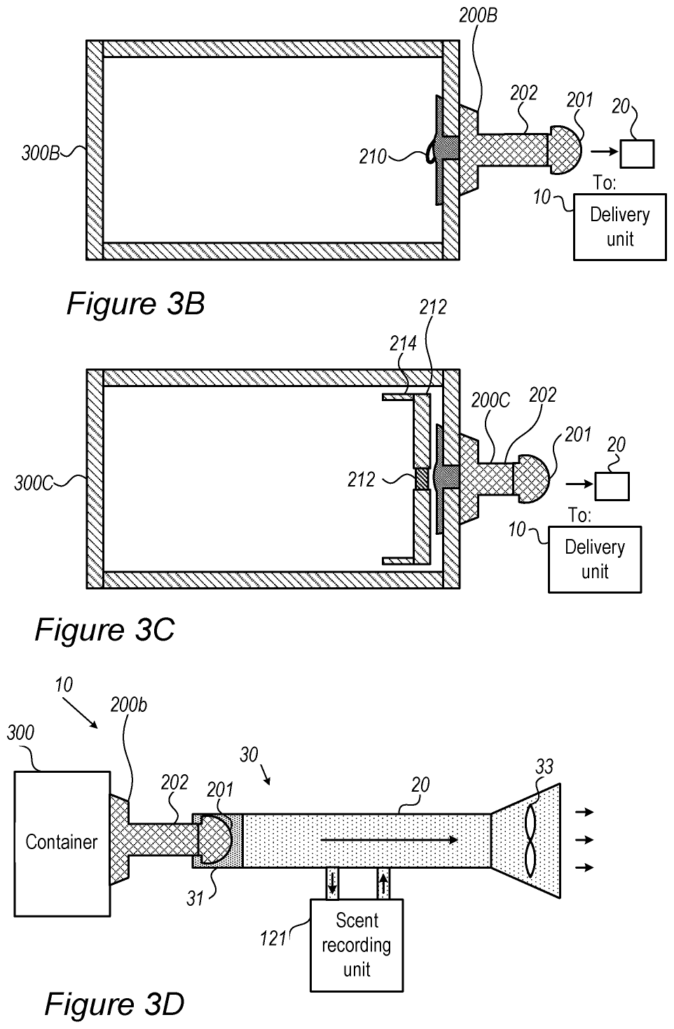

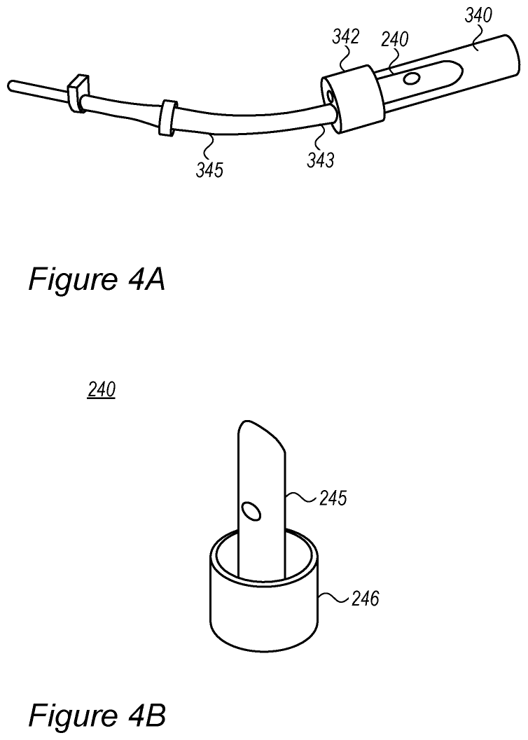

FIG. 1A is a high-level schematic block diagram of a system 100 for collecting and sensing volatile compounds according to some embodiments of the invention. System 100 may include a collecting unit 200 for collecting a source of VCs and a container 300 for containing the collected source of VCs. Some nonlimiting examples for collecting units 200A, 200B, 200C and 240 and containers 300A, 300B, 300C and 340 are given in FIGS. 3A-3D, and 4. System 100 may further include one or more scent recorder 120 and a delivery unit 10 for delivering a gas comprising VCs originated from the source, towards one or more scent recorders 120. In some embodiments, system 100 may further include a controller 400. In some embodiments, delivery system 10 may be configured (e.g., controlled by controller 400) to deliver a first portion of the gas containing the VCs, for ventilating at least part of delivery system 10 and to deliver a second portion of the delivered gas comprising the VCs for sensing the VCs by one or more scent recorders 120. In certain embodiments, controller 400 may be configured to regenerate one or more scent recorders 120 by creating a partial vacuum in its surrounding (e.g., in sensor chamber 40) and optionally heating one or more scent recorders 120.

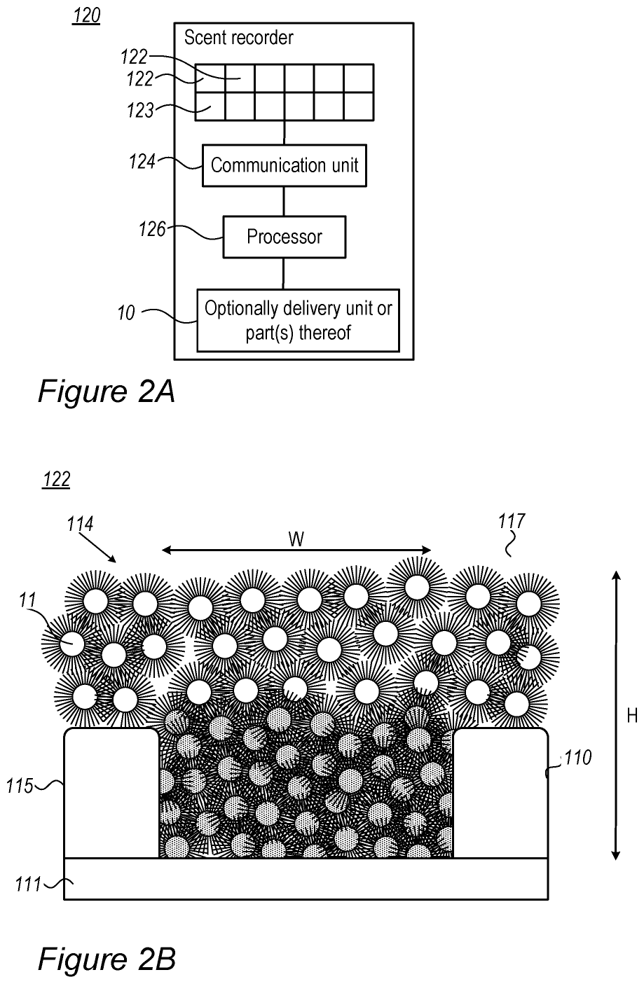

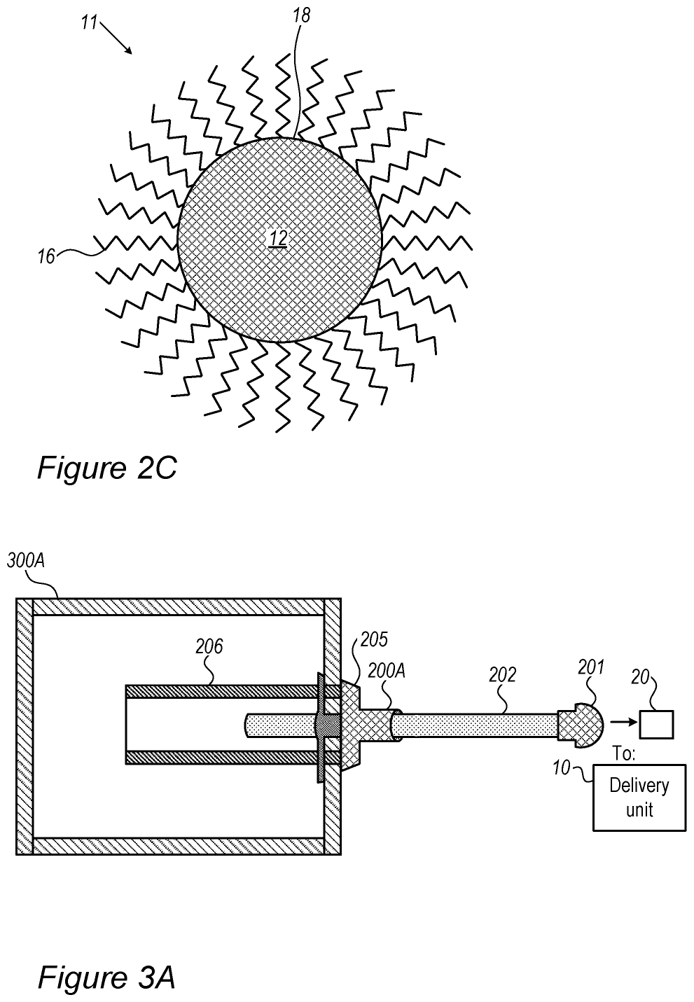

FIG. 1B is a high-level schematic illustration of a delivery system 10, according to some embodiments of the invention. Delivery system 10 may be configured to deliver gas containing the VCs to one or more scent recorders 120 configured to detect VCs. In some embodiments, system 10 may be controlled by a controller 400. One or more scent recorders 120 may include any sensor configured to produce a signal (e.g., an electric signal) when exposed to VCs in the gas phase. For example, one or more scent recorders 120 may include one or more chemi-resistors comprising metallic nanoparticles coated with organic ligands shell, as discussed in detail with respect to FIGS. 2A-2C. In other examples, one or more VCs' scent recorders 120 may include at least one of: metal oxide sensor (MOS), catalytic, near IR sensor, photoionization detector (PID), IR open path sensor, portable gas-chromatography mass spectrometer (GC-MS), electro-chemical sensor and the like. Delivery system 10 may comprise a manifold for distributing gases and delivering gas portions for sensing, as explained herein.

In some embodiments, delivery system 10 may further include an entrance tube/pipe 20 from which gas containing/carrying the VCs is introduced into delivery system 10. In some embodiments, delivery system 10 may further include a chamber 40 for holding one or more sensors 120. Chamber 40 may be configured to hold VCs 42 in the gas phase while exposing one or more scent recorders 120 to VCs 42. Chamber 40 may further include one or more additional sensors 60 for sensing an environment and the flow of gases inside chamber 40, for example, a thermometer, a barometer, a humidity sensor, a flowmeter and the like. In some embodiments, chamber 40 may further include a regeneration device 70 for regenerating one or scent recorders 120.

It is noted that various embodiments include various configurations of delivery unit 10, scent recorder 120 and scent recording unit 121. For example, delivery unit 10 and scent recording unit 121 may be configured as one unit (scent recording unit 121) including scent recorder 120, or delivery unit 10 may be separated from scent recording unit 121 functionally and/or physically, and be attachable thereto. Parts of delivery unit 10 may be attached to scent recorder 120 and configured as part of scent recording unit 121. Any of the elements illustrated in FIG. 1B in interaction with scent recorder 120 may be configured as part of scent recording unit 121, including parts or all of delivery unit 10. In certain embodiments, parts or all of delivery unit 10 may be part of container 300, with scent recording unit 121 being attachable thereto and including scent recorder 120, optionally with any of regeneration device 70, gas monitoring sensors 60, pump 36 etc. being part of scent recording unit 121. Accordingly, the terms scent recorder 120 and scent recording unit 121 may in certain embodiments used interchangeably, depending on the respective system configuration. The use of one of these terms herein rather than the other is therefore not limiting, and may be modified.

In some embodiments, the regeneration of delivery system 10, scent recorder(s) 120 and/or scent recording unit(s) 121 may be conducted by providing a first portion of the gas containing VCs 42 to respective one or more of delivery system 10, scent recorder(s) 120 and/or scent recording unit(s) 121 for flushing (e.g., cleaning) the respective surfaces from previously trapped VCs. In some embodiments, the first portion may be used for cleaning the pipes (e.g., pneumatics lines), valves, manifold etc. in delivery system 10, as well as possibly additional internal elements in scent recorder(s) 120 and/or scent recording unit(s) 121.

Regeneration device 70 may include a pump or a fan (e.g., pump 36 or fan 33) for pumping/blowing the first portion of the gas from container 300, 300A, 300B, 300C and 340 (illustrated in FIGS. 1A, 3A-3D and 4). Although the gas used for flushing and cleaning is not “clean” but rather contains VCs, it still may be used to ventilate the manifold. The inventors note that since the same gas is use for both cleaning and sensing, the final accuracy of the sensing may not be damaged or even be increased.

In certain embodiments, fan 33 may be used to blow the first portion of the gas to ventilate and/or replace gas in the volume of manifold 32 (illustrated schematically) and associated pipework of the pneumatic part of delivery system 10. Even though the first portion of the gas contains VCs, the inventors have found out that it still may be used to ventilate manifold 32. Pump 36 may be associated with scent recorder 120 (and/or scent recording unit 121) and be used to regenerate the corresponding sensors by creating a partial vacuum in their surroundings (e.g., with closed valve 34) and optionally heating the sensors. It is noted that while fan 33 typically denotes a high throughput device for replacing air in relatively large volumes using a small pressure difference (e.g., few tens of millibars), pump 36 typically denotes a device for creating larger pressure difference at smaller throughput, e.g., for creating relative vacuum in the surroundings of the sensors, typically operating at pressure differences approaching atmospheric pressure (e.g., hundreds of millibars), for example −0.8 bar yielding a vacuum of 0.2 bar (reduced pressure with respect to ambient 1 bar). Fan 33 typically moves a relatively large portion of gas for ventilating the respective pipework and units, while pump 36 typically moves a small volume of gas for sensing. It is noted that ventilating, or regenerating, part of the system may be carried out prior to the sensing as preparation, or after the sensing as preparation for the next measurement.

In some embodiments, regeneration device 70 may further include one or more tubes/pipes and/or valves for delivering the first portion of the gas toward one or more scent recorders 120, as disclosed herein below. In some embodiments, the flushing may occur due to a pressure differential between the container and the ambient. In some embodiments, fan 33 may blow the first portion of the gas containing the VCs over scent recorder 120 for flushing and cleaning the sensors (e.g., sensors 122 and 123 configured to sense VCs, non-limiting examples illustrated in FIG. 2A) of scent recorder 120 and pump 36 may be used for pumping the second portion of the gas containing the VCs used for sensing the VCs.

Additionally or alternatively, regeneration device 70 may include a heating element for heating one or more scent recorders 120, thus evaporating the VCs trapped by one or more scent recorders 120. In some embodiments, regeneration device 70 may include a vacuum pump for causing the evaporating the VCs due to sub-atmospheric pressure.

Regenerating sensor(s) for detecting VCs by creating the partial vacuum and optional heating may be carried out before the sensing is carried out, as preparation for the sensing, or after the sensing is carried out, as preparation for the next sensing. Accordingly, either a first portion of the VC-containing gas may be used for regenerating or flushing scent recorders 120 and a second portion of the VC-containing gas may be used for sensing the VCs, or the first portion of the VC-containing gas may be used for sensing the VCs while second portion of the VC-containing gas may be used for regenerating or flushing scent recorders 120 as preparation for the next measurement.

In certain embodiments, parts of the system may be cleaned or flushed with clean carrier gas, at least periodically, and the sensors for detecting VCs may be regenerated periodically irrespective of actual measurements.

Flushing—e.g., ventilating parts of the system and/or regenerating scent recorders—may be carried out with respect to parts or all of delivery unit 10 (e.g., filter 31, entrance tube/pipe 20, manifold 32 etc.), parts or all of scent detector(s) 120, and/or parts or all of container 300 and/or corresponding delivery pipe(s) 202 and related valve(s) 202, as described below. In certain embodiments, regeneration may comprise heating parts or all of delivery unit 10 (e.g., filter 31, entrance tube/pipe 20, manifold 32 etc.), parts or all of scent detector(s) 120, and/or parts or all of container 300 and/or corresponding delivery pipe(s) 202 and related valve(s) 202—to enhance evaporation of VCs.

In various embodiments, delivery of VC containing gas for sensing (by the sensors for detecting VCs in chamber 40 may be carried out actively, e.g., using fan 33 and/or pump 36 with open valve 34 and/or passively by creating a partial vacuum by pump 36 (with open valve 35 and closed valve 34) and then opening valve 34 to let VC-containing gas into sensor chamber 40.

In some embodiments, delivery system 10 may further include a gas circulation system 30 for directing VCs in a gas phase towards the one or more scent recorders 120 placed in chamber 40. Gas circulation system 30 may include: a fan 33, a pump 36, one or more gas monitoring sensors 60, one or more valves 34 and 35, a filter 31, a manifold 32, pipes 37 and the like. In should be understood by one skilled in the art that the component of a gas circulation system 30 illustrated in FIG. 1B are given as an example only, and a gas circulation system 30 according to embodiments of the invention may include some, all or more of the illustrated components. It is noted that any collection of parts of gas circulation system 30 may be ventilated by VC containing gas as disclosed herein.

Filter 31, possibly located at the entrance to pipe/tube 20 may be selected from a humidity trap, a dust filter, antiviral filter and the like. In some embodiments, at least one humidity trap included in filter 31 may reduce the level of humidity in the gas containing the VCs.

In some embodiments, delivery system 10 may include or may be in fluid connection with at least one container 300, 300A, 300B, 300C and 340 (illustrated in FIGS. 1A, 3A-3D and 4) for holding a source of VCs collected from at least one subject.

In some embodiments, controller 400 may be any computing device configured to execute instructions and method according to embodiments of the invention. Controller 400 may include a processor (e.g., a central processing unit processor (CPU), a graphics processing unit (GPU), a chip, a cloud based computing service or any suitable computing or computational device), a memory, a storage an input device, an output device, a communication unit and the like. In some embodiments, controller 400 may be configured to control pump 36 to pump the first portion of the gas containing the VCs prior to pumping the second portion of the gas, or control fan 31 to blow the first portion of the gas containing the VCs prior to pumping the second portion of the gas. For example, controller 400 may be configured to control fan 31 and\or pump 36 sequence to ensure that the sensing is of the VCs from container 300 and not from somewhere else such as environment or previously trapped VCs.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as a memory, computer-executable instructions such as an executable code and a controller. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices.

A system according to embodiments of the invention may include components such as, but not limited to, a plurality of central processing units (CPU), graphical processing units (GPUs), or any other suitable multi-purpose or specific processors or controllers (e.g., controllers similar to controller 400), a plurality of input units, a plurality of output units, a plurality of memory units, and a plurality of storage units. A system may additionally include other suitable hardware components and/or software components. In some embodiments, a system may include or may be, for example, a personal computer, a desktop, mobile phone, tablet, computer, a laptop computer, a workstation, a server computer, a network device, or any other suitable computing device.

In some embodiments, at least some of the components of controller 400 may be assembled on a printed circuit board (PCB) to be assembled inside unit 10. In some embodiments, unit 10 may further include a power supply 401 (illustrated in FIG. 1B) for providing electrical power to at least some of the components of delivery unit 10 and system 100, for example, computing device 400, scent recorders 120, sensors 60, pump 36, fan 31 and the like. In some embodiments, power supply 401 may be assembled on the PCB.

FIG. 2A is a high-level schematic illustration of scent recorder 120, according to some embodiments of the invention. Scent recorder 120 may include one or more sensors 122 and 123 configured to sense VCs, a communication unit (e.g., wireless or wired unit) 124 for communicating with a controller 400 (illustrated in FIGS. 1A and 1B) and a processor 126. Processor 126 (e.g., a chip) may collect readings from one or more sensors 122 and 123 and send them as a signal to controller 130 via communication unit 124.

Sensors 122 may be any sensor that may detect the presence of one or more volatile compounds (VCs), such as a VCs sensor and/or a chemiresistor sensor. For example, sensor 122 may include a material or structure that changes its electrical resistance in response to changes in the nearby chemical environment, for example, due to the presence of VCs. In some embodiments, sensor 122 may be a chemiresistor sensor for sensing VCs that may include a sensing element made from one of: carbon nanotubes, graphene, carbon nanoparticles, conductive polymers and the like. These chemiresistor sensors are sensitive to cleaning and regeneration cycles which are required after each measurement, due to the nonuniform nature of the sensor's material. Another optional chemiresistor sensor 122 may include conductive nanoparticles cores coated with organic ligands. In some embodiments, at least two sensors 122 may have substantially the same chemical composition and may differ in at least one physical attribute. In some embodiments, one or more sensors 123 may have a different chemical composition than sensors 122. It should be appreciated that scent recorder 120 may include an array of sensors all having the same chemical composition, such as sensors 122 or may include an array with two or more types of sensors, e.g., having different chemical composition. According to some embodiments, scent recorder 120 may include at least two sensors from each type of sensors in the array of sensors 122, 123.

FIG. 2B is a high-level schematic illustration of chemiresistor sensor 122, according to some embodiments of the invention. Chemiresistor sensor 122, in non-limiting examples for VCs sensors 122, 123, may include: two electrodes 110 and 115 and a sensing element 114. Electrodes 110 and 115 may be electrically connected to electrical connector(s) 111 (not insulated). Sensing element 114 may include a plurality of particles 11 (illustrated and discussed with respect to FIG. 2C). Particles 11 may be deposited between and/or on electrodes 110 and 115, using for example, ink jet printing. In some embodiments, the thickness H of sensing element 114 may vary between at least some of deposited sensors 122, 123 included in scent recorder 120. In some embodiments, the width W or the length (not illustrated) of the sensing region may vary between at least some of deposited sensors 122, 123 included in scent recorder 120. In some embodiments, the distribution or density of particles 11 may vary between at least some of deposited sensors 122, 123 included in scent recorder 120. For example, some of sensors 122, 123 may include sensing elements 114 having a first density or a second density or a combination the first and second densities, as illustrated schematically at the bottom and top parts of sensing element 114 in FIG. 2B. Any other number of different densities may appear in different sensors 122, 123.

FIG. 2C is a high-level schematic illustration of a particle 12 and ligands 16 in chemiresistor sensor 122, according to some embodiments of the invention. Chemiresistor sensor 122 may be used in non-limiting examples to implement VCs sensors 122, 123. In some embodiments, particle 11 may include a nanoparticle core 12. Nanoparticle core 12 may be made of a conductive material selected from a group consisting of: Ir, Ir-alloy, IrOₓ, Ru, Ru-alloy, RuOₓAu, Pt, Pd, Ag, Ni, Co, Cu, Al, Au/Ag, Au/Cu, Au/Ag/Cu, Au/Pt, Au/Pd, Au/Ag/Cu/Pd, Pt/Rh, Ni/Co, Pt/Ni/Fe and any combination thereof In some embodiments, nanoparticle core 12 may be made from any conductive metallic oxide. In some embodiments, the average diameter of nanoparticle core 12 may be of at most 100 nm, for example, 50 nm, 20 nm, 10 nm, 5 nm and 1 nm.

In some embodiments, nanoparticle core 12 may include a single metal or alloy and may have crystalline structure. In some embodiments, nanoparticle core 12 may include a single metal or alloy and may have an amorphous structure. In some embodiments, nanoparticle core 12 may include more than one metal or alloy, for example, a conductive oxide such as RuOₓ, IrOₓ and a metal, such as, Ru alloy, Ru, Ir alloy, Ir.

In some embodiments, particle 11 may further include a plurality of organic ligands 16 (illustrated schematically as zigzag lines) bonded, for example, via covalent bonds, from one side to the metallic core and capable of interacting with a VC. In some embodiments, organic ligands 16 are selected from a group consisting of: thiols, diazoniums, silanes, carboxylic acids, tri-chloro, methoxy, ethoxy, tri hydroxide, di-chloro, chloro and the like. In some embodiments, more than one type of organic ligand 16 may be bonded with one of core 12. For example, a core 12 that includes crystalline RuOx may be coated with organic ligands 16 of methoxy silane and trichloro silane. In some embodiments, the organic ligands can bond from one side, the side that includes the bonding group, for example, the chloro-silane, group with the surface of core 12 and from the other side with a VC, for example, a specific functional group can be added to the ligand chain that is configured to target (e.g., interact with) a specific VC. In some embodiments, the VC may interact with the branch of the organic ligands, for example, via VDW bonds, hydrogen bonds, Pi-Pi orbital interaction (for aromatics), dipole -dipole interaction (for polar), steric interference, charge to charge interaction, coordination bond, etc.

In some embodiments, nanoparticle core 12 may at least be partially covered with a conductive oxide layer 18 including at least one of: IrOₓ and RuOₓ or any other conductive oxides. In some embodiments, when nanoparticle cores 12 include crystalline or amorphous Ir, Ir-alloy, Ru or Ru-alloy, thin oxidation layer 18 may form on at least a portion of the surface of nanoparticle core 12. Thin oxidation layer 18 may be formed due to the exposure of nanoparticle cores 12 to air or oxygen. The thickness of oxidation layer 18 may be a few nanometers, for example, a native oxide layer of 1-2 nm. In some embodiments, oxidation layer 18 may allow stronger bonding between organic ligand 16 and the surface of cores 12.

FIGS. 3A-3D are high-level schematic illustrations of inflatable containers and collecting units for collecting a gas source of VCs, according to some embodiments of the invention. An inflatable container 300A (illustrated in FIG. 3A) may be made from any flexible and/or elastic material, such as an elastomer in fluid connection to a collecting unit 200A to allow gas containing the VCs to be inserted and held without leaking from container 300A. The material for container 300A (and also 300B and 300C) may be selected to be “VCs free”, meaning that the material does not emit VCs. Some examples for such materials may include, polypropylene, polyethylene, polyester, polytetrafluoroethylene and the like. Collecting unit 200A may include at least one nostril adapter 201 connected to at least one pipe 202 configured to deliver gas into container 300A via at least one, one directional valve 205 sleeve-like flexible structure 206 configured to allow entrance of air to container 300C until the container is filled with air in which flexible structure 296 is collapses and seal the entrance by the internal pressure of the gas in container 300A. At least one nostril adapter 201 may be configured to be inserted into the nostril or the mouth of a user for provision of air that includes VCs from the nostril or the mouth. At least one directional valve 205 may be configured to deliver the gas from at least one pipe 202 into container 300A while preventing gas from leaking or exiting form the container after the collecting of the gas. In some embodiments, at least one pipe 202 may be configured to be pushed towards the inner part of container 300A, thus breach the sealing of one directional valve 205, when there is a need to deliver the gas to be sensed by a scent recorder. In some embodiments, container 300A containing the gas containing the VCs may be stored for a predetermined time or immediately be connected to a delivery system 10, for example, via entrance pipe/tube 20. In some embodiments, entrance pipe/tube 20 (illustrated in FIG. 1B) is connectable to container 300A. For example, tube 20 may be configured to breach the sealing of container 300A, for example, by punching the container (e.g., using a needle, not illustrated) for extracting the gas containing the VCs form container 300A. For example, a user may exhale air for his/her mouth or nostril via adaptor 210 or directly to pipe 202 to be collected in container 300A. The gas containing VCs from the user's mouth may then be deliver by delivery system 11 to be sensed by scent recorder 120 (illustrated in FIGS. 1A, 1B and 2A). Such as analysis may result in determining at least one of: one or more types of VCs, a concentration, and a level of each type of VCs.

Reference is made to FIG. 3B which is an illustration of an inflatable container 300B and collecting unit 200B according to some embodiments of the invention. Inflatable container 300B (illustrated in FIG. 3B) may be made from any flexible and/or elastic material, such as an elastomer and may be connected to a collecting unit 200B. Collecting unit 200B may include at least one nostril adapter 201 connected to at least one pipe 202 configured to delivered gas into container 300B via at least one, one directional valve 210 having at least one membrane. In some embodiments, at least one pipe 202 may be used to breach the at least one membrane, thus allowing gas containing the VCs to be delivered to scent recorder 120 by delivery system 10.

Reference is made to FIG. 3C which is an illustration of an inflatable container 300C and collecting unit 200C according to some embodiments of the invention. Inflatable container 300C (illustrated in FIG. 3C) may be made from any flexible and/or elastic material, such as an elastomer and may be connected to a collecting unit 200C. Collecting unit 200C may include at least one nostril adapter 201 connected to at least one pipe 202 configured to deliver gas into container 300C via at least one, one directional valve 215. Valve 215 may include sleeve-like flexible structure 214 configured to allow entrance of air to container 300C until the container is filled with air in which flexible structure 214 is pushed and seal entrance 301 by the internal pressure of the gas in container 300C. Valve 215 may further include sealant film 212 (also known in the art as “easy-peel”), for sealing the entrance of air when the air containing the VCs is introduced into container 300B. Sealant film 212 may be breached if sufficient mechanical force is applied on the film, for example, when pipe 20 of delivery system 10 is inserted into container 300C, when the gas containing the VCs needs to be delivered to scent recorder 120.

In any of the illustrated embodiments, pipe(s) 202 from container 300 (and/or inflatable container 300A-C) may be attached to pipe 20 of delivery unit 10 and/or of scent recording unit 121 (when delivery system 10 is integrated as part thereof). As illustrated schematically in FIG. 3D, part(s) of delivery unit 10, whole delivery unit 10 and/or scent recording unit 121 as a whole may be attached to container 300 (and/or inflatable container 300A-C). For example, delivery system 10 may be part of the container assembly, and by attached to scent recording unit 121, e.g., at the level of valve 34 for optionally carrying out the flushing of scent recording unit 121 and for carrying our measurements by scent detector(s) 120 and/or the sensors for detecting VCs. In certain embodiments, delivery system 10 may include parts or all of collecting units 200 (or any of 200A, 200B and 200C), and optionally include all parts illustrated in FIG. 1B up to scent detector(s) 120 and/or even up to sensor(s) 122, 123, possibly including any of regeneration device 70, gas monitoring sensors 60, pump 36 etc. In various embodiments, flushing of delivery system 10 by the first portion of the collected gas may include any of the elements included in delivery system 10 in any of its configurations, or part(s) thereof.

In some embodiments, additional filters or valves may be included in collecting units 200A, 200B and 200C. For example, a humidity and/or dust filter (not illustrated) may be inserted in at least one nostril adapter 201 and/or to at least one pipe 202.

Reference is now made to FIGS. 4A and 4B which are illustrations of a nonlimiting examples for a container 340 and a collecting unit 240, according to some embodiments of the invention. Container 340 may include or may be made of any substantially rigid material (in comparison of the material of containers 300A, 300B, 300C and 300B) which may be configured to hold collecting unit 240 when inserted into container 340. Collecting unit 240 (illustrated in FIG. 4B) may include a collection component 245 to allow collection of samples, from solids and liquids and a threading 246 to seal container 340.

Container 340 may be in fluid connection with delivery unit, such as delivery unit 10, for example via pipe 345. Pipe 345 may be configured to connect to entrance pipe/tube 20. In some embodiments, delivery unit 10 may include a ventilation unit (e.g., a fan 33, pump 36, valves 34 and 35, etc.) for providing a carrier gas (e.g., air, nitrogen, argon, etc.) into container 340 to be exposed to the source of VCs and to carry the exposed gas towards one or more scent recorders 120. In some embodiments, collecting unit 240 may be designed to allow the carrier gas to flow effectively inside container 340 near or over the source of VCs to extract VCs from the source into the gas. In some embodiments, container 340 may include an entrance 342 for the introduction of the carrier gas and an exit 343 for delivering (e.g., blowing) the carrier gas carrying the VCs towards one or more scent recorders 120. In some embodiments, entrance 342 and exit 343 may lead to the same pipe 345, as illustrated schematically in FIG. 4A.

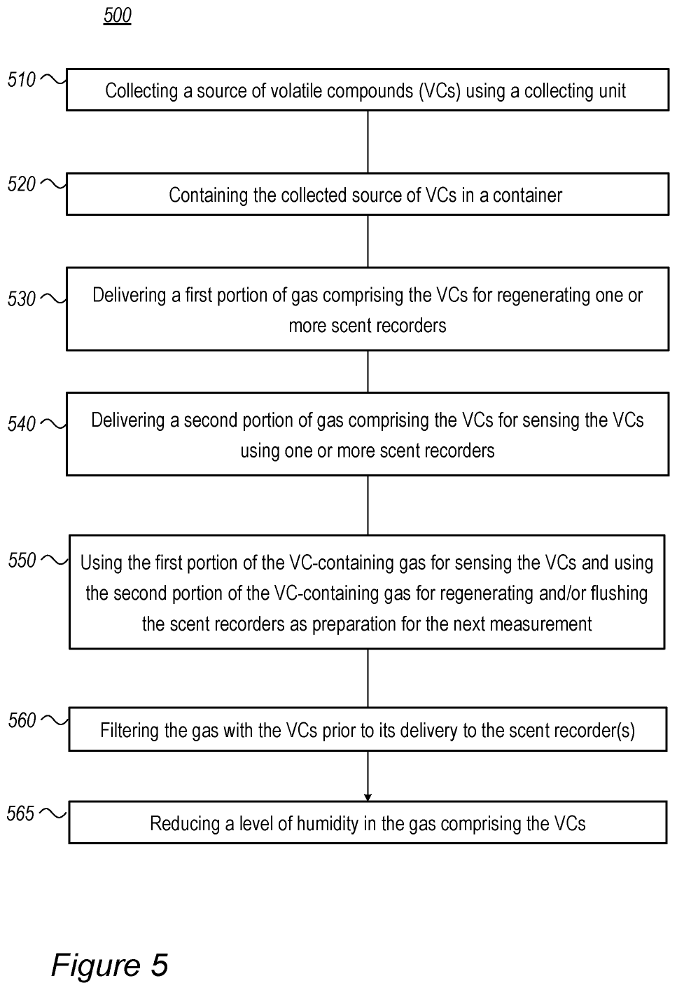

FIG. 5 is a high-level schematic flowchart of a method 500 of collecting and sensing volatile compounds, according to some embodiments of the invention. Method 500 may be performed by a system for collecting and sensing VCs, such as system 100, controlled by a controller, such as controller 400. In step 510, a source of VCs may be collected using a collecting unit. For example, collecting units 200A, 200B and 200C may be used for collected a gas containing the VCs (e.g., breathing air) and collecting unit 240 may be used for collecting solids and/or liquids.

In step 520, the collected source of VCs may be contained in a container. For example, gas containing VCs may be held in containers 300A, 300B and 300C and liquids and/or solids in container 340. In step 530, a first portion of gas containing the VCs may be delivered for ventilating at least part of the system, e.g., pipework and/or manifold(s) thereof. In some embodiments, if the source of VCs is not in a gas phase, for example solid or liquid, delivery system 10 may provide a carrier gas to container 340 to carry the VCs from the source. For example, pump 63 and/or fan 61 may pump and/or blow the first portion of gas containing the VCs form containers 300A, 300B, 300C or 340 to ventilate part(s) of the system, e.g., in order to flush and clean the scent recorder(s) from previously trapped VCs, and pump and/or blow a second portion of the gas may be delivered for sensing the VCs by one or more scent recorders 120.

In step 540, a second portion of gas comprising the VCs may be delivered to one or more scent recorders for sensing the VCs. For example, the second portion of gas carrying and/or containing the VCs may be delivered by delivery system 10 to one or more scent recorders 120 for sensing and/or detecting at least one of, one or more types of VCs, a concentration and a level of each type of VC, as discussed with respect to FIGS. 2A-2C.

In certain embodiments, the first portion of the gas may be blown, e.g., controllably, through at least part of the system (e.g., parts of the delivery system and possibly through the sensor chamber) prior to pumping the second portion of the gas to the one or more scent recorders for sensing. Regeneration of the scent recorders may be carried out before the measurement or after the measurement as preparation for the next measurement.

Method 500 may comprise regenerating the sensor(s) for detecting VCs by creating the partial vacuum and optionally heating the sensor chamber—may be carried out before or after the sensing. For example, method 500 may comprise using the first portion of the VC-containing gas for regenerating or flushing the scent recorders and the second portion of the VC-containing gas for sensing the VCs, or method 500 may comprise using the first portion of the VC-containing gas for sensing the VCs and using the second portion of the VC-containing gas for regenerating and/or flushing the scent recorders as preparation for the next measurement (stage 550).

In certain embodiments, method 500 may further comprise filtering the gas comprising the VCs prior to the delivery of the gas to the one or more scent recorder (stage 560) and/or reducing a level of humidity in the gas comprising the VCs (stage 565).

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A system for collecting and sensing volatile compounds (VCs), the system comprising:
a collecting unit for collecting a source of VCs;
a container for containing the collected source of VCs;
one or more scent recorders comprising at least one sensor configured to sense VCs; and
a delivery unit for delivering a gas comprising VCs collected from the source, via the container towards the one or more scent recorders,
wherein a first portion of the delivered gas unit is used for regenerating at least part of the one or more scent recorders, and a second portion of the delivered gas unit is used for sensing the VCs by the one or more scent recorders.

2. (canceled)

3. The system of claim 1, wherein the one or more scent recorders is configured to detect at least one of: one or more types of VCs, a concentration of one or more types of VCs, and a level of one or more types of VCs.

4. The system according to claim 1, wherein the source of VCs is a gas containing the VCs and wherein the container is an inflatable container.

5. The system of claim 4, wherein the collecting unit includes at least one one-directional valve for delivering the gas containing the VCs into the inflatable container.

6. The system of claim 4, wherein the delivery unit includes a tube connectable to the inflatable container.

7. The system according to claim 1, wherein the source of VCs is liquid and/or solid and the collecting unit is configured to place the source of VCs inside the container.

8. The system of claim 7, wherein the delivery unit includes a ventilation unit configured to: introduce a carrier gas into the container, expose the carrier gas to the source of VCs, and deliver the exposed gas towards the one or more scent recorders.

9. The system of claim 8, wherein the container includes an entrance for the introduction of the carrier gas and an exit for delivering the exposed carrier gas towards the one or more scent recorders.

10. The system according to claim 1, wherein the delivery unit further comprises: at least one fan configured to blow the first portion of the gas containing the VCs to ventilate at least part of the delivery unit, and at least one pump configured to deliver the second portion of the gas containing the VCs to the at least one sensor in the one or more scent recorders.

11. The system of claim 10, further comprising a controller configured to control the at least one fan to blow the first portion of the gas prior to the pumping of the second portion of the gas to the one or more scent recorders.

12. The system of claim 10, wherein the controller is further configured to regenerate the one or more scent recorders by creating a partial vacuum in a chamber holding the at least one sensor configured to sense VCs, and optionally heating the at least one sensor for detecting VCs.

13. The system according to claim 1, further comprising at least one filter for filtering the gas comprising the VCs prior to the delivery of the gas to the one or more scent recorder.

14. The system according to claim 1, further comprising at least one humidity trap for reducing a level of humidity in the gas comprising the VCs.

15. A method of collecting and sensing volatile compounds (VCs) by a corresponding system, the method comprising:
collecting a source of VCs and accumulating gas comprising the VCs;
delivering a first portion of the gas comprising the VCs for regenerating at least part of the system;
delivering a second portion of the gas comprising the VCs to one or more scent recorders that comprise at least one sensor configured to sense VCs; and
sensing the VCs in the second portion of the gas using the one or more scent recorders, wherein the at least one sensor and/or the one or more scent recorders is regenerated prior to or after the sensing.

16. The method of claim 15, wherein the sensing comprises detecting at least one of: one or more types of VCs, a concentration of the one or more types of VCs, and a level of the one or more types of VCs.

17. The method of claim 15, further comprising: exposing a carrier gas to the source of VCs, and carrying the exposed gas towards the one or more scent recorders.

18. The method of claim 17, further comprising blowing, controllably, the first portion of the gas through the at least part of the system prior to delivering the second portion of the gas to the one or more scent recorders, wherein the delivering is carried out by blowing or pumping.

19. The method according to claim 15, further comprising filtering the gas comprising the VCs prior to the delivery of the gas to the one or more scent recorder.

20. The method according to claim 15, further comprising reducing a level of humidity in the gas comprising the VCs.
